(19) United States
(12) Patent Application Publication
(10) Pub. No.: US 2006/0165756 A1
(43) Pub. Date: Jul. 27, 2006

(54) Method for weight management

(76) Inventors: Steven J. Catani, Athens, GA (US); Steven D. Clarke, North Brunswick, NJ (US); Dondeena G. Bradley, New York, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/044,462

(22) Filed: Jan. 27, 2005

(51) Int. Cl. G06Q 10/00; A61K 47/00; G06Q 50/00

(52) U.S. Cl. 424/439; 128/898; 705/2

(57) ABSTRACT

The present invention is directed to a method for managing body weight by measuring preload efficiency and having the energy load of a food serving and providing information as to the measured preload efficiency and energy load of the selected food serving to enable an informed choice of an appropriate food serving. The method can optionally further include the step of calculating a net preload effect that is based on the measured preload efficiency and a known or estimated energy load for the selected food serving.

### SUMMARY OF THE INVENTION

The present invention is directed to a method for managing body weight by providing the consumer with the overall effect on energy intake of a food product. This overall effect includes not only the energy content of the food, but also the effect the food will have on subsequent meals. The method includes determining the effect of a food consumed prior to the next meal on the calories consumed at that meal “the preload efficiency” and determining the energy load of the food serving and providing information as to the measured preload efficiency and energy load of the selected food serving to enable an informed choice of an appropriate food serving. The method can optionally further include the step of calculating a net preload effect that is based on the measured preload efficiency and a known or estimated energy load for the selected food serving.

### BACKGROUND

Several methods have been developed to communicate the energy content of food. These methods provide a person seeking to lose, maintain, or gain weight with a way to monitor their energy intake and set their intake relative to their energy expenditure appropriately.

The most common method of communication is the nutrient content labels found on all packaged foods and beverages. These labels not only include energy content reported in calories (actually kilo-calories) but also the amount of macronutrients and other key components. These labels allow consumers to not only control their calorie intake, but also to manage their consumption of fats, carbohydrates, proteins, and other nutrients.

Restaurant menus often contain the energy content of the various meals available. Fast food restaurants often post the nutrient content of their various food choices. These communication tools sometimes also include more detailed nutrient content information similar to what appears on packaged foods and beverages.

Some organizations such as Weight Watchers use patented point systems to easily communicate the energy content of a meal or food choice. This system allows the consumer to simply count the number of points in a day to manage their energy input. U.S. Pat. No. 6,040,531 describes a process for controlling body weight in which selection of food servings is based on a calculated point value and a range of allotted daily points that are adjusted for weight change. The calculated point value is a function of measured calories, total fat and dietary fiber for serving sizes specified in readily estimable units. U.S. Pat. No. 4,652,241 describes a system for providing a plurality of independent, readily identifiable, movable members, each of which represents a pre-defined food group and portion and positioning the movable members in cooperative association with meal designating zones, a unique, highly flexible food consumption, planning and control system is achieved. The plurality of movable members comprise in their entirety, all of the food to be consumed by an individual in one day, and the meal designating zones identify all of the meals to be eaten by that individual during one day. By moving each food designating member from an intake designating zone to a meal designating zone, for each food group and portion consumed during that meal, the individual can quickly and easily control and record the food to be consumed, as well as develop diverse, varied meals within the desired maximum caloric intake allowed.

In the 1990s, the fat content of food became important and consumers began using fat content (usually expressed in grams) as a means to control their energy intake from fat. Likewise in the late 1990s, Dr. Atkins' book on the benefits of low carbohydrate diets caused consumers to begin to pay special attention to energy consumption from carbohydrates. Again, consumers used information on the nutrition labels to manage carbohydrate input. Other means for communication also resulted from the “low carb” trend to allow consumers to discern the type of carbohydrates. These new measures, such as “net carbs” and “impact carbs”, provided specific information on effect on blood sugar that the meal or food item would have. Published U.S. Patent Application No. 2004/0043106 describes a method of determining a standard comestible equivalent glycemic load of a dietary comestible by establishing a reliable glycemic response index for a standard comestible wherein the index correlates glycemic response with glycemic load, determining the glycemic response produced by a dietary comestible and identifying the standard comestible glycemic load from the index which is correlated with the glycemic response of the dietary comestible.

Organizations for people with special dietary needs, such as the American Diabetics Association, have developed point systems for use in simply monitoring glucose and glucose precursor intake.

Several methods to use the energy contents of foods and meals have been disclosed. These provide a means to capture and report energy intake over a period. Some also record energy expenditure and provide information about the balance between input and output.

Other devices help users determine the content of foods and meals through electronic databases or via measurement devices such as volumetric vessels or scales. Published U.S. Patent Application No. 2003/0187683 describes a weight control software system that may generate meal plans that are automatically updated based on the updated weight of the user following the weight control program. U.S. Pat. No. 5,729,479 describes a multifunctional diet calculator that is operative to provide prompts to a user in addition to keeping track of food intake. The prompts may be useful in ensuring that the user consumes desired amounts of substances, such as water or pills, which are helpful in ensuring success of a diet or other regime. Still others provide a means to calculate body metrics such as resting metabolic rate, an important piece of information for calculating a balance energy information.

Some patents, which have addressed weight control, include U.S. Pat. No. 5,398,688, U.S. Pat. No. 5,233,520, and U.S. Pat. No. 4,652,241. The method described in the Laniado (US '688) attempts to limit a dieter's daily food intake by clinically monitoring the dieter over a period of time, establishing and measuring a physiological variable to generate a relationship between that variable and that of the dieter's rate and/or amount of food ingested, calculating a maximum eating time to ingest a pre-determined amount of food, and providing a indicator, such as an alarm, to the dieter to stop eating when the eating time has lapsed.

The method disclosed in Kretsch (US '520) provides an interactive computerized dietary measurement system that measures weight, nutrient value, and dietary component of all food items stored in the computer system database. It is used to collect, process, and summarize all dietary information for the dieter so that the dieter may maintain a proper nutritional dietary regimen.

The system of McCarty (US '241) is a daily planner that includes a listing of a dieter's daily pre-determined intake and permits the dieter to manipulate any individual food group/item to any pre-selected mealtime. The dieter can eat all the foods selected for that day in any sequence preferred.

Other devices have been disclosed that allow users to make use of other nutrient input information such as carbohydrate input. These devices utilize the measurement of blood glucose either directly or indirectly through spectrometric means to manage glucose metabolism.

Many investigators have disclosed the effect of a snack, beverage, or other type of food input prior to the next meal. The “preloads” often have a profound effect on the meal consumption at the subsequent meals. Some studies suggest this effect is highly dependent on the nutrient content of the preload. For example, Rolls et al. found that nutrient composition had a differential effect on hunger, fullness and food intake. “The Specificity of Satiety: The Influence of Foods of Different Macronutrient Content on the Development of Satiety,” Physiology & Behavior, Vol. 43, pp. 145-153.

Some studies have found that a glass of water has virtually no effect on the subsequent meal while a sucrose-sweetened beverage lowers next meal consumption significantly. See, for example, Rolls et al., “Effects of Drinks Sweetened with Sucrose or Aspartame on Hunger, Thirst and Food Intake in Men,” Physiology & Behavior, Vol. 48, pp. 19-26.

The degree of compensation varies not only with macronutrient content, but also with delivery vehicle and phase. Some compounds can cause the effect with virtually no caloric content. It is clear that absent ignoring these effects can cause consumers trying to manage their weight to make incorrect snack, beverage and meal choices. The present invention describes a program that overcomes these shortcomings as well as others.

### BRIEF DESCRIPTION OF THE INVENTION

The present invention is directed to a method for managing body weight using a preload efficiency of a food serving and a known, measured or estimated energy load of the same or similar food serving. Information is provided comprising the preload efficiency and energy load of the selected food serving to enable an informed choice by the consumer of a food serving. The preload efficiency and energy load can be provided in a single communication to the consumer. Alternatively, the information can be provided to the consumer on a net basis, wherein the net basis is calculated by multiplying the preload efficiency by the energy load. A net preload effect can be determined based on the measured preload efficiency and an energy load for the selected food serving.

The information can be communicated in numeric, alphabetic or symbolic form or combinations thereof. For example, the preload efficiency can be expressed as a numeric value to the extent the food serving is compensated for by a reduced energy intake at a subsequent meal. A net preload effect can be expressed as a numeric value for an expected reduction in energy intake at a subsequent meal. The information can be expressed as a symbol or letter that communicates a value for which the food serving is compensated for by a reduction in energy intake at a subsequent meal. Alteratively, the information can be expressed as a color that communicates the percentage for which the food serving is compensated for by a reduction in energy intake at a subsequent meal. Still further, the information can be expressed as a symbol or letter that communicates a percentage for which the preload is compensated for by a reduction in energy intake at a subsequent meal. The information can be expressed as a color that communicates the percentage for which the food serving is compensated for by a reduction in energy intake at a subsequent meal. The information can be provided on a product label for the selected food serving.

### DETAILED DESCRIPTION OF THE INVENTION

The present invention includes processes for controlling the body weight of animals, particularly human beings. Control of body weight may involve weight reduction, weight gain, or current weight maintenance.

Applicants have developed a method and a communication system that provides consumers with information needed to account for the preload effect in making food choices. The method comprises quantifying the physical effect of ingesting a selected food serving as a preload serving relative to a subsequent meal using a standardized method (“satiating impact”) and labeling the selected food serving or a substantially equivalent food serving with the measured effect.

The satiating impact can be measured by reference to a foodstuff as consumed or by measuring a reference foodstuff from which a preload effect can be extrapolated/estimated for a foodstuff to be consumed. The satiating impact can be expressed in terms of the satiated energy load, as a percentage of the foodstuff energy load that translates into a satiated energy load, or both. Preload efficiency means, for purposes of this application, the percentage for which a preload serving is compensated for by energy intake reduction at an immediately subsequent meal. A net preload effect or satiating impact is based on the measured preload efficiency times the known or estimated energy load for a selected preload serving.

The net preload effect or satiating impact can be communicated or displayed to individuals via a visual or auditory code. The preload can be pre-made or pre-packaged, or made by the consumer using a provided recipe, and can take the form of a snack, a beverage, a meal, a medical food, a food for special dietary purpose, a dietary supplement such as a pill, a chew, a dissolvable strip, a beverage, or a beverage mix, or combinations of said food products.

The information on energy load is preferably communicated in terms of grams of macronutrients, calories or kilocalories (kcal), joules or kilo joules, btus, or other standard method of measuring energy content.

The information on the preload efficiency, energy load, and/or preload effect can alternatively be communicated or displayed, for example, using numeric, alphabetic, symbolic, graphical, color-coded or auditory representations and can appear as part of a recipe, or on a product label.

The code can reflect: a) the percentage for which the preload is compensated for by energy intake reduction at a subsequent meal; b) the energy reduction expected at the next meal; c) the net energy for the preload, i.e. the energy contained in the food serving less the energy reduction expected at the next meal. Other variations of this theme are readily apparent to those of ordinary skill in the art.

The code can be the numerical value of calorie reduction at the next meal or a simple code, such as the amount of “Weight Watcher”™ points which will be offset by the preload. The numerical value could be expressed in terms of percentage points or a numerical scale that correlates to percentage points.

The information can also include a method to communicate the period of effectiveness after the preload is consumed and the variability in the effect over a population, and variation of impact over time. The preload efficiency and corresponding preload effect can be determined as they relate to a packaged and labeled food product or with respect to a reference food product from which values can be estimated or extrapolated for a given food product.

The method can be used as a part of a weight loss plan, a weight maintenance plan, or a weight gain plan.

The effect of the preload, the preload efficiency, can be measured by any means known to those skilled in the art. The energy load for food servings are published and means are well known for determining and verifying such results. One means for measuring preloading efficiency has been developed by S. V. Kirkmeyer and R. D. Mattes and reported in the International Journal of Obesity (2000), Vol. 24, pp. 1167-1175, in an article entitled “Effects of Food Attributes on Hunger and Food Intake.” The methodology requires the calculation of: ((normal energy intake from a self-selected diet+energy from the preload)−(actual energy intake from self-selected meal+energy of preload))/(energy value of preload). The calculation is the proportion of energy provided by the preload that was offset by an adjustment in the self-selected diet.

The normal energy intake from a self-selected diet was based on a prior analysis of subject's dietary habits. Each subject was trained to keep free-feeding diaries and allowed to follow their uncontrolled regular (though recorded) eating routine. Subjects then participated in the study that included preload foodstuff periods followed by a self-selected meal.

An alternative means for measuring preloading efficiency is shown in an article by Rolls et al., “The Specificity of Satiety: The Influence of Foods of Different Macronutrient Content on the Development of Satiety,” Physiology & Behavior, Vol. 43, pp. 145-153. The preloads used in the study consisted of equicaloric amounts of foods that were high in one macronutrient and low in other macronutrients, and one food that was of a mixed macronutrient composition. The caloric content of each preload was approximately 300 Kcal. The preloads were made up to 390 grams in weight in a way that each subject received an equivalent weight of preload. The self-selection meal was offered two hours after the preload snack. The foods available in the self-selection meal were chosen so that the macronutrient selection following the preloads could be assessed. For the analysis of food intake, the weight of food consumed and energy intake were calculated with and without the inclusion of fluid intake and the calories contributed by a regular soft drink.

Other variants will be clear to those skilled in the art.

We claim:

1. A method for managing body weight comprising:
(a) determining preload efficiency of a food serving; and
(b) having a known, measured or estimated energy load of the same or similar food serving and
(c) providing information comprising the preload efficiency and energy load of the selected food serving to enable an informed choice by the consumer of a food serving.

2. The method according to claim 1, wherein the preload efficiency and energy load are provided in a single communication to the consumer.

3. The method according to claim 1, wherein the information is provided to the consumer on a net energy input basis.

4. The method according to claim 3, wherein the net energy basis is calculated by multiplying the preload efficiency by the energy load.

5. The method according to claim 1, wherein a net preload effect is determined based on the measured preload efficiency and an energy load for the selected food serving.

6. The method according to claim 1, wherein the selected food serving is a snack, a beverage, a meal, a medical food, a food for special dietary purpose, a dietary supplement, or combinations thereof.

7. The method according to claim 6, wherein the supplement is a pill, a chew, a dissolvable strip, a beverage, or a beverage mix.

8. The method according to claim 1, wherein the food serving is pre-made or pre-packaged.

9. The method according to claim 1, wherein the consumer makes the food serving from a recipe.

10. The method according to claim 1, wherein the energy load is communicated in grams of macronutrients, calories or kilocalories, joules or kilo joules, btus, or other standard method of measuring energy content.

11. The method according to claim 1 wherein the information is communicated in numeric, alphabetic or symbolic form or combinations thereof.

12. The method according to claim 1 wherein preload efficiency is expressed as a numeric value to the extent the food serving is compensated for by a reduced energy intake at a subsequent meal.

13. The method according to claim 1 wherein the information further comprises a net preload effect that is expressed as a numeric value for an expected reduction in energy intake at a subsequent meal.

14. The method according to claim 1 wherein the information is expressed as a symbol or letter that communicates a value for which the food serving is compensated for by a reduction in energy intake at a subsequent meal.

15. The method according to claim 1 wherein the information is expressed as a color that communicates the percentage for which the food serving is compensated for by a reduction in energy intake at a subsequent meal.

16. The method according to claim 1 wherein the information is expressed as a symbol or letter that communicates a percentage for which the preload is compensated for by a reduction in energy intake at a subsequent meal.

17. The method according to claim 1 wherein the information is expressed as a color that communicates the percentage for which the food serving s compensated for by a reduction in energy intake at a subsequent meal.

18. The method according to claim 1 wherein the information is provided on a product label for the selected food serving.

19. The method according to claim 1 that further comprises the step of communicating the variability over time of the preload effect after consumption.

20. The method according to claim 1 further comprising the step of expressing the variability in the preload efficiency or preload effect over a sample population.

21. The method in claim 1 which is part of a weight loss plan.

22. The method in claim 1 which is a part of a weight maintenance plan.

23. The method in claim 1 which is part of a weight gain plan.

24. The method of claim 1 that is designed for an animal other than human.

25. The method of claim 1 that is designed for a human.
